(19) United States
(12) Patent Application Publication
(10) Pub. No.: US 2026/0216005 A1
(43) Pub. Date: Jul. 30, 2026

(54) MEDICATION MANAGEMENT ALERT DEVICES, SYSTEMS, AND METHODS OF USE THEREOF

(71) Applicant: SALUTEMP CORP., Dover (DE)

(72) Inventors: Theodora Amalia Christopher, Durham, NC (US); Benjamin Paul Dottinger, Sparta, NJ (US)

(21) Appl. No.: 19/108,019

(22) PCT Filed: Sep. 1, 2023
(86) PCT No.: PCT/US2023/031885

§ 371 Date: Feb. 28, 2025

Related U.S. Application Data
(60) Provisional application No. 63/403,428, filed on Sep. 2, 2022.

(51) Int. Cl. A61J 7/04; G16H 40/40; G16H 40/67

(52) U.S. Cl. CPC A61J 7/0481; G16H 40/40; G16H 40/67; A61J 2200/72; A61J 2200/74

(57) ABSTRACT

Medication management devices are disclosed. The device includes a housing constructed and arranged to receive a container of medication, one or more sensors, and a power source. The medication management device is capable of monitoring one or more environmental parameters using the one or more sensors. The medication management device includes a communication module, e.g., a wireless communication module, e.g., a BLUETOOTH® radio. Medication management systems are disclosed. The system includes a medication management device and a controller operatively connected to the medication management device. The device includes a housing constructed and arranged to receive a container of medication, one or more sensors, and a power source, with the device being capable of monitoring one or more environmental parameters. The controller is operatively connected to the one or more sensors. Methods of managing the use of a medication using the devices and systems are also disclosed.

### CROSS-REFERENCE TO RELATED APPLICATIONS

This application claims priority to U.S. Patent Application No. 63/403,428, filed Sep. 2, 2022, the entire contents of which are incorporated herein by reference in their entirety.

### BACKGROUND

The present invention relates to systems and methods of use for medication compliance. Proper medication storage and prescription adherence is vital for any medication to work properly. Of the top-selling biotech drugs worldwide in 2019, temperature-sensitive drugs make up six of the top 10 in terms of revenue in billions of US dollars. Temperature-sensitive drugs made up a total of 47.3 billion USD in 2019. Temperature-sensitive medications, e.g., biologics, are commonly used in hospitals, nursing homes and elderly facilities. However, home-use of biologics is becoming increasingly common as the cost of these medications goes down and doctors prescribe them more often than shelf-stable medications because of their increased efficacy. This efficacy, and even sometimes medication safety for home-use, decreases as medication use and storage compliance is less controlled. For example, hospitals and other care facilities generally store all biologic medications in dedicated medication refrigerators or freezers whereas home users of biologic medications don't often have the means or knowledge for proper storage. In general, at least 80% of a medication regimen must be completed for any medical efficacy to be attained. However, roughly only 20% of outpatient medication users take their full prescription regimen according to doctor instructions.

There is a need for a medication alert management system that is capable of alerting the user and potential third parties of improper medication storage conditions and user adherence to a medication schedule. There is further a need for such a system as described above that is capable of use with various medication storage systems, such as bottles, blister packs, and a cabinet or compartment for the storage of multiple medications.

### SUMMARY

The present disclosure relates to devices, systems, and methods for providing and using a medication storage management system. The system as described herein may provide users with alerts for when a medication is stored improperly and also information about proper storage and how to mitigate improper storage conditions. The present disclosure may also be configured to send the user, medication provider or other chosen third parties alerts when the patient fails to follow the medication regimen. The proper storage thresholds may be determined by the pharmacist or pharmaceutical distributor, with such thresholds added to the system via barcode or manual entry. The present disclosure is also portable and moveable from one medication bottle to another, or it may be applied to a shelf or cabinet to monitor the storage of multiple medications. The present disclosure is also capable of use with a variety of medication containers, e.g., injector pens, pill bottles, blister packets, and the like.

In accordance with an aspect, there is provided a medication management device. The device includes a housing constructed and arranged to receive a container of medication. The device includes one or more sensors. The device further includes a power source. The device, e.g., the medication management device, is capable of monitoring one or more environmental parameters.

In some embodiments, the one or more environmental parameters include temperature, light, pressure, relative humidity, oxygen content, or a combination thereof. For example, in certain embodiments, the one or more environmental parameters include temperature, light, pressure, relative humidity, or a combination thereof. In other embodiments, the one or more environmental parameters include temperature, light, relative humidity, or a combination thereof.

In some embodiments, the one or more sensors include a thermometer, a hygrometer, a light sensor, a pressure sensor, or a combination thereof. The sensors are disposed on any suitable location of the device. For example, the one or more sensors are disposed on a lower portion of the device. In certain embodiments, the one or more sensors are disposed on a side wall of the device.

In some embodiments, the power source includes a connection to an electrical receptacle. In some embodiments, the power source includes a battery, such as a rechargeable battery, e.g., charged using a USB connector or over a wireless charging protocol, e.g., Qi charging.

In certain embodiments, the device is constructed and arranged to be releasably attached to a container, e.g., bottle, of a medication. For example, a container of medication may fit, e.g., by friction, into an appropriately sized recess in the device. In other embodiments, the device is constructed and arranged to substantially enclose one or more containers of medication, e.g., within an interior volume. For example, the device may include an insert, e.g., a removable insert, that is dimensioned to secure one or more medications within the interior volume of the device.

In further embodiments, the device includes a communication module configured to collect and transmit data from the one or more sensors.

In accordance with an aspect, there is provided a medication management device. The device includes a housing constructed and arranged to receive a container of medication. The device includes one or more sensors. The device further includes a communication module. The device additionally includes a power source. The device, e.g., the medication management device, is capable of monitoring one or more environmental parameters.

In some embodiments, the communication module includes a wireless communications module. The wireless communication module can include a wireless radio or emitter, such as a Wi-Fi radio, BLUETOOTH® radio, 5G NR FR2 radio, or other wireless transmission standard, e.g., based on the LTE Cat 1, LTE Cat M1 or Cat NB1 standard. In specific embodiments, the wireless communication module includes a BLUETOOTH® radio. In some embodiments, the wireless communication module includes both a BLUETOOTH® radio and a Wi-Fi radio.

In further embodiments of any device disclosed herein, the housing includes a securing mechanism. The securing mechanism includes a magnetic switch, e.g., a Reed switch, operatively coupled to a sensor board of the device.

In some embodiments, the device may be adapted to hold an injector pen medication delivery system. The injector pen may be a hormone pen, such as an epinephrine injector pen or an insulin injector pen.

In further embodiments, the device may include an insert dimensioned to fit within the housing constructed and arranged to hold the container of medicine.

In some embodiments, the device may have length of about 25 mm to about 300 mm. In particular embodiments, the device may be about 200 mm long, i.e., about eight inches long.

In some embodiments, the device may have a width of about 25 mm to about 300 mm. In particular embodiments, the device may be about 64 mm wide, i.e., about 2.5 inches wide.

In some embodiments, the device may have a thickness or height of about 25 mm to about 300 mm. In particular embodiments, the device may be about 64 mm high or thick, i.e., about 2.5 inches high or thick.

In certain embodiments, the device may have a length of about 200 mm, a width of about 64 mm, and a height or thickness of about 64 mm, i.e., a length of about eight inches, a width of about 2.5 inches, and a height or thickness of about 2.5 inches.

In accordance with an aspect, there is provided a medication management system. The medication management system includes a medication management device and a controller. The medication management device includes a housing constructed and arranged to receive a container of medication. The medication management device includes one or more sensors. The medication management device further includes a power source. The controller operatively connected to the one or more sensors of the medication management device. The medication management device is capable of monitoring one or more environmental parameters.

In some embodiments, the medication management device and the controller communicate wirelessly, e.g., Wi-Fi, BLUETOOTH®, 5G NR FR2, or other wireless transmission standard, e.g., based on the LTE Cat 1, LTE Cat M1 or Cat NB1 standard. In some embodiments, the medication management device and the controller communicate over a wired connection, e.g., USB, RS-232, RJ-11, RJ-45, i.e., Ethernet, or another wired standard.

In some embodiments, the controller includes a non-transitory computer-readable medium storing instructions which, when executed by the controller, provide an interface configured to display data pertaining to measurements from the one or more sensors.

In some embodiments, the controller is configured with a dosing schedule for a medication. For example, the controller is configured to provide an alert to a user, e.g., a third party, upon detecting a deviation from the medication dosing schedule. In some embodiments, the controller is configured to provide an alert to a user based on a measurement from the one or more sensors exceeding a predetermined threshold.

In some embodiments, the controller is configured to permit access to data pertaining to measurement from the one or more sensors and/or deviations from the medication dosing schedule by one or more third parties. The one or more third parties may be provided and alert from the controller upon detection of a deviation from the medication dosing schedule or based on a measurement from the one or more sensors exceeding a predetermined threshold.

In further embodiments, the system includes an external communication module operatively coupled to one or both of the medication management device and the controller. The external communications module may be a wireless communications module. The wireless communication module can include a wireless radio or emitter, such as a Wi-Fi radio, BLUETOOTH® radio, 5G NR FR2 radio, or other wireless transmission standard, e.g., based on the LTE Cat 1, LTE Cat M1 or Cat NB1 standard. In specific embodiments, the wireless communication module includes a Wi-Fi radio. In some embodiments, the wireless communication module includes both a BLUETOOTH® radio and a Wi-Fi radio.

In accordance with an aspect, there is provided a method of managing the use of a medication. The method includes providing a medication management system including a medication management device and a controller. The medication management device includes one or more sensors, and the controller includes a user interface. The method includes interfacing one or more containers of medication within the medication management device. The method further includes monitoring one or more environmental parameters of the medication management device.

In further embodiments, the method includes configuring the controller with a dosing schedule for the medication. In further embodiment, the method includes configuring the controller with predetermined threshold values for measurements from the one or more sensors.

In some embodiments, the method includes providing an alert to a user upon detection of a deviation from the medication dosing schedule. The alert provided to the user may be based on a measurement from the one or more sensors exceeding the predetermined threshold. For example, the user interface of the controller displays information pertaining to the deviation from the medication dosing schedule or measurement from the one or more sensors exceeding the predetermined threshold to the user.

In further embodiments, the method includes providing an alert to one or more third parties based on a measurement from the one or more sensors exceeding the predetermined threshold. In further embodiments, the method includes providing an alert to one or more third parties based on upon detection of a deviation from the medication dosing schedule.

In any embodiment of a method disclosed herein, the medication management device is connectable to an individual container of medication.

In any embodiment of a method disclosed herein, the medication management system is constructed and arranged to store one or more containers of medication.

### BRIEF DESCRIPTION OF THE DRAWINGS

FIG. 1 illustrates a central monitor system, according to an embodiment.

FIG. 2 illustrates a sensor board including labeling of the different components of the sensor board, according to an embodiment. In FIG. 2, J is a jack or connector, SW is a switch connector, C is a capacitor, R is a resistor, BT are battery terminals, Y is a magnetostriction oscillator, and U are integrated circuits (ICs) including the one or more sensors.

FIG. 3 illustrates a wireless communications platform, according to an embodiment.

FIG. 4 illustrates a central monitor system, according to another embodiment. In FIG. 3, the central monitor system includes the housing embodied as a clamshell enclosure, a magnetic circuit for the clamshell latch, a sensor board integrated into the housing, and a connector for a power supply.

FIG. 5 illustrates a method of managing the use of a medication, according to an embodiment.

### DETAILED DESCRIPTION

The present disclosure provides devices and systems useful, e.g., for the monitoring of a medication, and related methods. In some embodiments, the devices and systems are used to alert a user, and optionally, one or third parties, e.g., pharmacists, doctors, or family members, as to the status of a medication. For example, the devices and systems described herein may provide information regarding whether a medication is being stored improperly and being used in compliance with directives. The devices and systems described herein may further provide information regarding the physical parameters of the medication storage conditions, for example, one or more of temperature, light exposure, and relative humidity. In some embodiments, the devices and systems include a pressure sensor to measure adherence to a medication schedule, e.g., by measuring a change in mass of a container of a medication or the opening and closing of a housing storing the medication. In some embodiments, the user and optional third parties are able to set a medication schedule within the system, e.g., on a controller, where the system will alert the user and third parties when a medication is missed or taken outside of a predetermined schedule.

### Definitions

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the” include their corresponding plural references unless the context clearly dictates otherwise.

The terms “acquire” or “acquiring.” as used herein, refer to obtaining possession of a value, e.g., a numerical value, or image, or a physical entity, e.g., a sample, by “directly acquiring” or “indirectly acquiring” the value or physical entity. “Directly acquiring” means performing a process, e.g., performing an analytical method or protocol, to obtain the value or physical entity. “Indirectly acquiring” refers to receiving the value or physical entity from another party or source, e.g., a third-party laboratory that directly acquired the physical entity or value. Directly acquiring a value or physical entity includes performing a process that includes a physical change in a physical substance or the use of a machine or device. Examples of directly acquiring a value include obtaining a reading or sensor information from a device described herein.

The term “device,” as used herein, refers to an entity, e.g., an enclosure, capable of storing a medication. A device may include a housing, one or more sensors, and one or more power sources. A device may be made of any material, such as metal or, plastic. In an embodiment, the device is sized appropriately to house a medication. In an embodiment, the device is durable and can withstand multiple rounds of storage. The device herein is also referred to as a “medication management device.” In an embodiment, the device may include a securing mechanism, e.g., a latch, e.g., a magnetic latch, whose actuation is detectable by any included electrical components of the device.

The term “subject” or “user,” as used herein, refers to a human or non-human animal. In an embodiment, the subject is a human, i.e., a male or female, e.g., of any age group, a pediatric subject, e.g., infant, child, adolescent, or adult subject, e.g., young adult, middle-aged adult, or senior adult. In an embodiment, the subject is a non-human animal, for example, a mamma, e.g., a mouse, a dog, a primate, e.g., a cynomolgus monkey or a rhesus monkey. In an embodiment, the subject is a commercially relevant mammal, e.g., a cattle, pig, horse, sheep, goat, cat, or dog, or a bird, e.g., a commercially relevant bird such as a chicken, duck, goose, or turkey. In certain embodiments, the animal is a mammal. The animal may be a male or female and at any stage of development. A non-human animal may be a transgenic animal.

The term “system,” as described herein, refers to a device. e.g., a device described herein, as well as any peripheral hardware or software, including a controller. In an embodiment, a system comprises a device and a controller capable of reporting/providing information on the status of a medication to a user. A system may comprise a single device or a plurality of devices. The system herein is also referred to as a “medication management system.”

### Devices

The present disclosure provides devices and systems for use in managing medication storage and use compliance. In some embodiments, the device as described herein comprises one or more sensors for measuring parameters for medication storage and compliance. In some embodiments, the one or more sensors include a thermometer, a light meter, a hygrometer for measuring relative humidity, a trace gas sensor, and a pressure sensor. In some embodiments, the pressure sensor is a combined pressure-weight sensor. In some embodiments, the one or more sensors are pre-calibrated and data-logging. In some embodiments, the group of sensors are operatively connected to a controller, where said controller collects data from the various sensors. In some embodiments, the device may include a communication module connected to the one or more sensors that includes a memory capable of storing the data from measurements by the one or more sensors. The communication module is connected to the controller and can transmit said data to the controller. The combination of the one or more sensors on the device and the controller may be referred to as a medication management system.

In some embodiments, the controller includes an application for interaction with a user, e.g., a patient or provider. In some embodiments, the controller further comprises a rechargeable battery as a power source. In some embodiments, the rechargeable battery can be charged using a wired connection, such as a Universal Serial Bus (USB) connection; alternatively, or in addition, the rechargeable battery may be charged using a wireless charging system, e.g., a Qi coil or the like. The device may incorporate an integrated cable for charging, such as a USB cable that can be connected to a standard power inverter, i.e., terminated with a USB Type-A or Type-C connector. Alternatively, or in addition, the device may include a separate USB cable for charging the device, e.g., a Micro-USB, Mini-USB, or USB Type-C cable, that connects to the appropriate connector on the device. The device may include a charging status indicator, such as a screen, light, or other display, that communicates when the device is low on power, charging, and/or fully charged. In some embodiments, the controller comprises a wired power source, with a rechargeable battery as a backup in the case of a power failure. In some embodiments, the one or more sensors may communicate with the controller via wired transmission protocol, such as broadband internet connection, e.g., a Local Area Network (LAN) or a Wide Area Network (WAN). The one or more sensors further may include appropriate wired connections for data transmission under any suitable standard, e.g., RS-232, USB, or the like. Alternatively, or in addition, the one or more sensors may communicate with the controller wirelessly, for example, by means of the GSM cellular telephone network using a modem connected to a computer or a hand-held device, by Wi-Fi, BLUETOOTH®, 5G NR FR2, or other wireless data collection methods known in the art, e.g., based on the LTE Cat 1, LTE Cat M1 or Cat NB1 standard. The controller may be standalone platform, e.g., a desktop computer. Alternatively, the controller may be a mobile platform, e.g., a cellular telephone, a tablet, a laptop computer, and the like. In some embodiments, the device may have a length of about 25 mm to about 300 mm, e.g., about 25 mm to about 300 mm, about 30 mm to about 275 mm, about 35 mm to about 250 mm, about 40 mm to about 225 mm, about 45 mm to about 200 mm, about 50 mm to about 175 mm, about 55 mm to about 150 mm, about 60 mm to about 125 mm, or about 70 mm to about 100 mm. In some embodiments, the device may have a length of about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, about 110 mm, about 111 mm, about 112 mm, about 113 mm, about 114 mm, about 115 mm, about 116 mm, about 117 mm, about 118 mm, about 119 mm, about 120 mm, about 121 mm, about 122 mm, about 123 mm, about 124 mm, about 125 mm, about 126 mm, about 127 mm, about 128 mm, about 129 mm, about 130 mm, about 131 mm, about 132 mm, about 133 mm, about 134 mm, about 135 mm, about 136 mm, about 137 mm, about 138 mm, about 139 mm, about 140 mm, about 141 mm, about 142 mm, about 143 mm, about 144 mm, about 145 mm, about 146 mm, about 147 mm, about 148 mm, about 149 mm, about 150 mm, about 151 mm, about 152 mm, about 153 mm, about 154 mm, about 155 mm, about 156 mm, about 157 mm, about 158 mm, about 159 mm, about 160 mm, about 161 mm, about 162 mm, about 163 mm, about 164 mm, about 165 mm, about 166 mm, about 167 mm, about 168 mm, about 169 mm, about 170 mm, about 171 mm, about 172 mm, about 173 mm, about 174 mm, about 175 mm, about 176 mm, about 177 mm, about 178 mm, about 179 mm, about 180 mm, about 181 mm, about 182 mm, about 183 mm, about 184 mm, about 185 mm, about 186 mm, about 187 mm, about 188 mm, about 189 mm, about 190 mm, about 191 mm, about 192 mm, about 193 mm, about 194 mm, about 195 mm, about 196 mm, about 197 mm, about 198 mm, about 199 mm, about 200 mm, about 201 mm, about 202 mm, about 203 mm, about 204 mm, about 205 mm, about 206 mm, about 207 mm, about 208 mm, about 209 mm, about 210 mm, about 211 mm, about 212 mm, about 213 mm, about 214 mm, about 215 mm, about 216 mm, about 217 mm, about 218 mm, about 219 mm, about 220 mm, about 221 mm, about 222 mm, about 223 mm, about 224 mm, about 225 mm, about 226 mm, about 227 mm, about 228 mm, about 229 mm, about 230 mm, about 231 mm, about 232 mm, about 233 mm, about 234 mm, about 235 mm, about 236 mm, about 237 mm, about 238 mm, about 239 mm, about 240 mm, about 241 mm, about 242 mm, about 243 mm, about 244 mm, about 245 mm, about 246 mm, about 247 mm, about 248 mm, about 249 mm, about 250 mm, about 251 mm, about 252 mm, about 253 mm, about 254 mm, about 255 mm, about 256 mm, about 257 mm, about 258 mm, about 259 mm, about 260 mm, about 261 mm, about 262 mm, about 263 mm, about 264 mm, about 265 mm, about 266 mm, about 267 mm, about 268 mm, about 269 mm, about 270 mm, about 271 mm, about 272 mm, about 273 mm, about 274 mm, about 275 mm, about 276 mm, about 277 mm, about 278 mm, about 279 mm, about 280 mm, about 281 mm, about 282 mm, about 283 mm, about 284 mm, about 285 mm, about 286 mm, about 287 mm, about 288 mm, about 289 mm, about 290 mm, about 291 mm, about 292 mm, about 293 mm, about 294 mm, about 295 mm, about 296 mm, about 297 mm, about 298 mm, about 299 mm, or about 300 mm.

In certain embodiments, the device has a length of about 50-500 mm, i.e., a length of about 1-20 inches. In certain embodiments, the device has a length of about 100-300 mm. In certain embodiments, the device has a length of about 100 mm. In certain embodiments, the device has a length of about 200 mm. In certain embodiments, the device has a length of about 300 mm.

In some embodiments, the device may have a width of about 25 mm to about 300 mm, e.g., about 25 mm to about 300 mm, about 30 mm to about 275 mm, about 35 mm to about 250 mm, about 40 mm to about 225 mm, about 45 mm to about 200 mm, about 50 mm to about 175 mm, about 55 mm to about 150 mm, about 60 mm to about 125 mm, or about 70 mm to about 100 mm. In some embodiments, the device may have a width of about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, about 110 mm, about 111 mm, about 112 mm, about 113 mm, about 114 mm, about 115 mm, about 116 mm, about 117 mm, about 118 mm, about 119 mm, about 120 mm, about 121 mm, about 122 mm, about 123 mm, about 124 mm, about 125 mm, about 126 mm, about 127 mm, about 128 mm, about 129 mm, about 130 mm, about 131 mm, about 132 mm, about 133 mm, about 134 mm, about 135 mm, about 136 mm, about 137 mm, about 138 mm, about 139 mm, about 140 mm, about 141 mm, about 142 mm, about 143 mm, about 144 mm, about 145 mm, about 146 mm, about 147 mm, about 148 mm, about 149 mm, about 150 mm, about 151 mm, about 152 mm, about 153 mm, about 154 mm, about 155 mm, about 156 mm, about 157 mm, about 158 mm, about 159 mm, about 160 mm, about 161 mm, about 162 mm, about 163 mm, about 164 mm, about 165 mm, about 166 mm, about 167 mm, about 168 mm, about 169 mm, about 170 mm, about 171 mm, about 172 mm, about 173 mm, about 174 mm, about 175 mm, about 176 mm, about 177 mm, about 178 mm, about 179 mm, about 180 mm, about 181 mm, about 182 mm, about 183 mm, about 184 mm, about 185 mm, about 186 mm, about 187 mm, about 188 mm, about 189 mm, about 190 mm, about 191 mm, about 192 mm, about 193 mm, about 194 mm, about 195 mm, about 196 mm, about 197 mm, about 198 mm, about 199 mm, about 200 mm, about 201 mm, about 202 mm, about 203 mm, about 204 mm, about 205 mm, about 206 mm, about 207 mm, about 208 mm, about 209 mm, about 210 mm, about 211 mm, about 212 mm, about 213 mm, about 214 mm, about 215 mm, about 216 mm, about 217 mm, about 218 mm, about 219 mm, about 220 mm, about 221 mm, about 222 mm, about 223 mm, about 224 mm, about 225 mm, about 226 mm, about 227 mm, about 228 mm, about 229 mm, about 230 mm, about 231 mm, about 232 mm, about 233 mm, about 234 mm, about 235 mm, about 236 mm, about 237 mm, about 238 mm, about 239 mm, about 240 mm, about 241 mm, about 242 mm, about 243 mm, about 244 mm, about 245 mm, about 246 mm, about 247 mm, about 248 mm, about 249 mm, about 250 mm, about 251 mm, about 252 mm, about 253 mm, about 254 mm, about 255 mm, about 256 mm, about 257 mm, about 258 mm, about 259 mm, about 260 mm, about 261 mm, about 262 mm, about 263 mm, about 264 mm, about 265 mm, about 266 mm, about 267 mm, about 268 mm, about 269 mm, about 270 mm, about 271 mm, about 272 mm, about 273 mm, about 274 mm, about 275 mm, about 276 mm, about 277 mm, about 278 mm, about 279 mm, about 280 mm, about 281 mm, about 282 mm, about 283 mm, about 284 mm, about 285 mm, about 286 mm, about 287 mm, about 288 mm, about 289 mm, about 290 mm, about 291 mm, about 292 mm, about 293 mm, about 294 mm, about 295 mm, about 296 mm, about 297 mm, about 298 mm, about 299 mm, or about 300 mm.

In certain embodiments, the device has a width of about 10-200 mm. In certain embodiments, the device has a width of about 10-100 mm. In certain embodiments, the device has a width of about 50-100 mm. In certain embodiments, the device has a width of about 50-75 mm. In certain embodiments, the device has a width of about 60 mm. In certain embodiments, the device has a width of about 65 mm. In some embodiments, the device may have a thickness or height of about 25 mm to about 300 mm, e.g., about 25 mm to about 300 mm, about 30 mm to about 275 mm, about 35 mm to about 250 mm, about 40 mm to about 225 mm, about 45 mm to about 200 mm, about 50 mm to about 175 mm, about 55 mm to about 150 mm, about 60 mm to about 125 mm, or about 70 mm to about 100 mm. In some embodiments, the device may have a thickness or height of about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, about 100 mm, about 101 mm, about 102 mm, about 103 mm, about 104 mm, about 105 mm, about 106 mm, about 107 mm, about 108 mm, about 109 mm, about 110 mm, about 111 mm, about 112 mm, about 113 mm, about 114 mm, about 115 mm, about 116 mm, about 117 mm, about 118 mm, about 119 mm, about 120 mm, about 121 mm, about 122 mm, about 123 mm, about 124 mm, about 125 mm, about 126 mm, about 127 mm, about 128 mm, about 129 mm, about 130 mm, about 131 mm, about 132 mm, about 133 mm, about 134 mm, about 135 mm, about 136 mm, about 137 mm, about 138 mm, about 139 mm, about 140 mm, about 141 mm, about 142 mm, about 143 mm, about 144 mm, about 145 mm, about 146 mm, about 147 mm, about 148 mm, about 149 mm, about 150 mm, about 151 mm, about 152 mm, about 153 mm, about 154 mm, about 155 mm, about 156 mm, about 157 mm, about 158 mm, about 159 mm, about 160 mm, about 161 mm, about 162 mm, about 163 mm, about 164 mm, about 165 mm, about 166 mm, about 167 mm, about 168 mm, about 169 mm, about 170 mm, about 171 mm, about 172 mm, about 173 mm, about 174 mm, about 175 mm, about 176 mm, about 177 mm, about 178 mm, about 179 mm, about 180 mm, about 181 mm, about 182 mm, about 183 mm, about 184 mm, about 185 mm, about 186 mm, about 187 mm, about 188 mm, about 189 mm, about 190 mm, about 191 mm, about 192 mm, about 193 mm, about 194 mm, about 195 mm, about 196 mm, about 197 mm, about 198 mm, about 199 mm, about 200 mm, about 201 mm, about 202 mm, about 203 mm, about 204 mm, about 205 mm, about 206 mm, about 207 mm, about 208 mm, about 209 mm, about 210 mm, about 211 mm, about 212 mm, about 213 mm, about 214 mm, about 215 mm, about 216 mm, about 217 mm, about 218 mm, about 219 mm, about 220 mm, about 221 mm, about 222 mm, about 223 mm, about 224 mm, about 225 mm, about 226 mm, about 227 mm, about 228 mm, about 229 mm, about 230 mm, about 231 mm, about 232 mm, about 233 mm, about 234 mm, about 235 mm, about 236 mm, about 237 mm, about 238 mm, about 239 mm, about 240 mm, about 241 mm, about 242 mm, about 243 mm, about 244 mm, about 245 mm, about 246 mm, about 247 mm, about 248 mm, about 249 mm, about 250 mm, about 251 mm, about 252 mm, about 253 mm, about 254 mm, about 255 mm, about 256 mm, about 257 mm, about 258 mm, about 259 mm, about 260 mm, about 261 mm, about 262 mm, about 263 mm, about 264 mm, about 265 mm, about 266 mm, about 267 mm, about 268 mm, about 269 mm, about 270 mm, about 271 mm, about 272 mm, about 273 mm, about 274 mm, about 275 mm, about 276 mm, about 277 mm, about 278 mm, about 279 mm, about 280 mm, about 281 mm, about 282 mm, about 283 mm, about 284 mm, about 285 mm, about 286 mm, about 287 mm, about 288 mm, about 289 mm, about 290 mm, about 291 mm, about 292 mm, about 293 mm, about 294 mm, about 295 mm, about 296 mm, about 297 mm, about 298 mm, about 299 mm, or about 300 mm.

In certain embodiments, the device has a height or thickness of about 64 mm, i.e., a height or thickness of about 2.5 inches. In certain embodiments, the device has dimensions of about 200 mm L by about 64 mm W by about 64 mm high/thick.

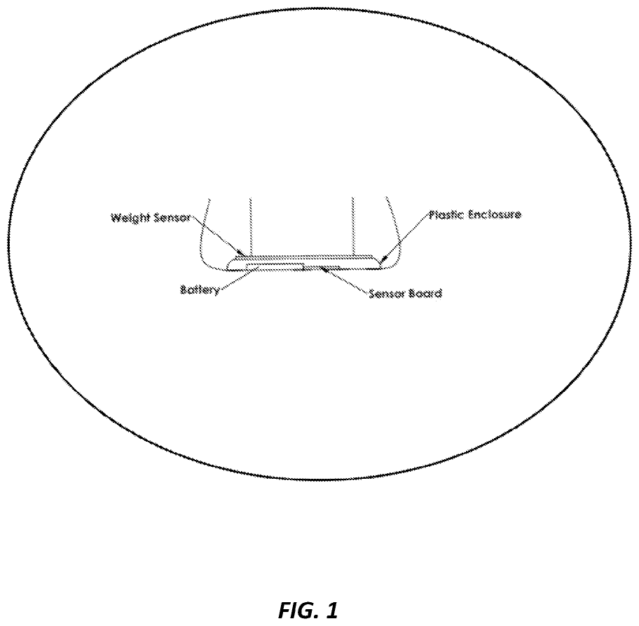

FIG. 1 is an illustration of the device described herein. The system comprises a weight/pressure sensor, a battery, a sensor board having the one or more sensors, and an enclosure for housing the battery and sensor board. The sensor collects data each time the container of medication is removed from and placed back onto the device. In some embodiments, the sensor board includes one or more of a thermometer, a light meter, a hygrometer, or any combination thereof. In some embodiments, the device is constructed and arranged to be releasably connected to a container, e.g., a medication container. For example, the device may be sized to be affixed, e.g., snapped on and off, to a container of medicine. Alternatively, the device may be sized such that the container of medicine can be rested on the sensor without touching any other part of the device, e.g., the device remains in its location and only the container of medicine is moved. For example, the device may include a hinged housing, such as a clamshell-style case or a cabinet-type enclosure, that can be opened by a user and has the sensor board and the one or more sensor integrated therein. In other embodiments, the device constructed and arranged to store a container within a larger housing, such as a refrigerator, cabinet, or the like, and a container of medication placed onto the sensor. In some embodiments, the pressure/weight sensor is disposed on a bottom or underside of the device. In specific embodiments, the thermometer, hygrometer and light meter are disposed on the bottom or underside of the device. In other embodiments, the thermometer, hygrometer and light meter are disposed on interior sides of the device.

When the device is in a hinged, i.e., clamshell, configuration, one or both halves of the device may be made from a material that is resistant to impact and environmental changes. For example, one or both halves of the device may be made from a metal, e.g., steel, brass, bronze, aluminum, or zinc. In some embodiments, one or both halves of the device may be made from a finished wood, e.g., alder, balsa, beech, hickory, mahogany, maple, oak, teak, walnut, bamboo, cedar, Douglas fir, juniper, pine, redwood, spruce, and yew. In some embodiments, one or both halves of the device may be made from a polymer, e.g., polyethylene terephthalate (PETE or PET), polyethylene, polyvinyl chloride (PVC), polypropylene, polystyrene, polylactic acid, polycarbonate, acrylic, acetal, polyether ether ketone (PEEK), polyoxymethylene, nylon, acrylonitrile butadiene styrene (ABS), and other common polymers and blends thereof. In some embodiments, both halves of the hinged device may be made from the same material, i.e., both halves made from a polymer. Alternatively, one half of the hinged device may be made from one material and the other half of the hinged device may be made from a different material. In some embodiments, the device may be made from a pliable material, i.e., leather or other heavier fabric.

In some embodiments, an inner surface of the device, independent of the material used to fabricate the device, includes a surface coating. This surface coating may be used to prevent scratches to the medication containers within the inner volume of the device. Further, an inner coating can provide a layer of thermal insulation to the inner volume of the device, absorb an accidental release of a medication within the inner volume of the device, and protect a user's hands or fingers when accessing the medication within the inner volume of the device. Common surface coatings can include, but are not limited to, absorbent coatings, flocked coatings, various fabric linings, i.e., microfiber or microsuede, moisture and stain repellant coatings, i.e., TEFLON® and SCOTCHGUARD®, and other similar surface treatments.

In some embodiments, the device may include an insert dimensioned to sit within the device that is sized to hold one or more containers of medicine. For example, when the device is in a hinged or clamshell configuration, the device may include an insert having outer dimensions to fit within the volume of one-half of the clamshell including one or more cutouts that are sized to receive containers of medicine. In a non-limiting embodiment, the insert for the device may include a cutout that is dimensioned to receive any standard injector pen. In some embodiments, injector pen delivered medicines suitable for storage within the devices disclosed herein include hormone injector pens, e.g., epinephrine, i.e., an EPIPEN®, a growth hormone, e.g., somatropin, an insulin injector pen, or a peptide-based medicine, e.g., GLP-1 analogs, e.g., dulaglutide, semaglutide, liraglutide, and exenatide, among others. In another non-limiting embodiment, the insert for the device may include a plurality of cylindrical openings that are dimensioned to receive vials of a medication, such as insulin vials, vials of a biologic, i.e., antibody based medicine. The insert and any cutouts therein for one or more containers of medicine can have any suitable shape that provides for proper fitment within the device such that the insert cannot be accidentally removed from the device and the one or more container of medicine are secured within the insert when installed in the device. The insert may be made from any suitable material that can provide support and protection to the one or more containers of medicine, such as hard polymer, a soft polymer, a foam, e.g., a urethane foam, e.g., memory foam, a sponge, or any other suitable material that can be shaped appropriately.

In some embodiments, the device is a base where a container of medication is placed upon, such as that illustrated in FIG. 1. In this configuration, the base includes the necessary electrical components within a recess and is designed to be placed onto a shelf in a storage system, such as a cabinet, refrigerator, or the like. In use, a user can place (and subsequently remove) one or more containers of medication onto the base after accessing the location where the based is stored. The base may be made from a metal, e.g., steel, brass, bronze, aluminum, or zinc. In some embodiments, the device may be made from a finished wood, e.g., alder, balsa, beech, hickory, mahogany, maple, oak, teak, walnut, bamboo, cedar, Douglas fir, juniper, pine, redwood, spruce, and yew. In some embodiments, the device may be made from a polymer, e.g., polyethylene terephthalate (PETE or PET), polyethylene, polyvinyl chloride (PVC), polypropylene, polystyrene, polylactic acid, polycarbonate, acrylic, acetal, polyether ether ketone (PEEK), polyoxymethylene, nylon, acrylonitrile butadiene styrene (ABS), and other common polymers and blends thereof.

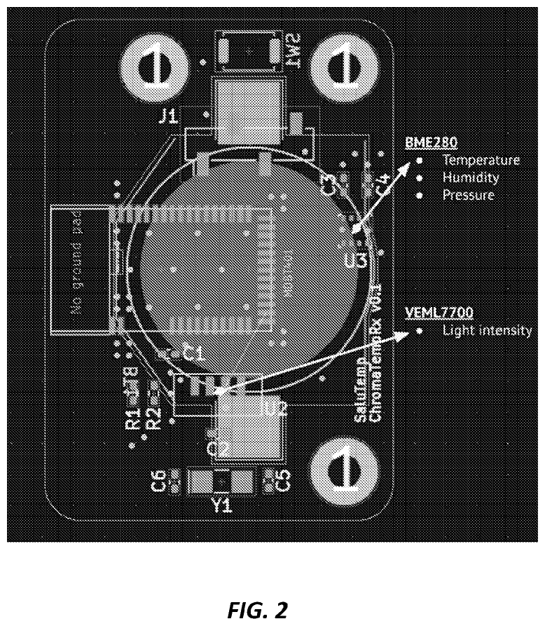

FIG. 2 illustrates an embodiment of a sensor board suitable for incorporation into a device disclosed herein. With reference to FIG. 2, the sensor board includes resistors R1 and R2, capacitors C1, C2, C3, C4, C5, and C6, switch SW1, battery terminals BT1, and jack connector J1. Further illustrated in FIG. 2 is magnetostriction oscillator Y1 that is used to determine the opening and closing of the device. In this configuration, the securing mechanism, e.g., latch or clasp, of the device may be a magnetic switch, e.g., a Reed switch. In a Reed switch, two metallic blades are spaced apart to provide for a discontinuity in an electrical circuit. When a permanent magnet is applied to the metallic blades, the blades come into contact, thereby completing the electrical circuit. When the magnet is moved away from the metallic blades, the blades return to their default separation to again provide for a discontinuity in the electrical circuit. This cycle of continuity and discontinuity in a magnetic-induced electrical circuit can serve as a metric for the opening and closing of the device. While the securing mechanism, e.g., latch or clasp, of the device has been described as a magnetic circuit, this is only an embodiment and the securing mechanism, e.g., latch or clasp, of the device can be any suitable latch or clasp that can provide an indication to an end user that the device has been opened or closed.

With continued reference to FIG. 2, the sensor board includes one or more integrated circuits U2 and U3, each incorporating one or more sensors. The one or more sensors can each be a dedicated integrated circuit. Alternatively, an integrated circuit can include one or more sensors on one package. As illustrated in FIG. 2, integrated circuit U2 includes a light sensor and integrated circuit U3 is a combination IC package including sensors for temperature, relative humidity, trace gases, and pressure. It is noted that these integrated circuits are only illustrative embodiments, and this disclosure is not limited by the specific sensor ICs, their type, or their arrangement on the sensor board.

In some embodiments, the light sensor that is incorporated into the device is a photoelectric sensor, taking advantage of the photoelectric effect. In a photoelectric sensor, photons from a light source impact the sensor's active area, ejecting electrons from the active area. The ejected electrons register a detectable current, with the current change from zero, i.e., darkness, being indicative of the amount of light received by the sensor. When installed in the devices disclosed herein, the light sensor is triggered when photons from the ambient environment strike the active area of the sensor, such as when the device is opened to permit access to the one or more containers of medicine stored therein. Light sensors suitable for use in devices disclosed herein may have an illuminance range of about 0 lux to about 6000 lux on a linear scale, with 1 lux being equivalent to 1lumen/m². Zero lux is complete darkness, consistent with the inside of the device when closed and 6000 lux is consistent with the device being fully opened inside of a brightly lit indoor room.

In some embodiments, the one or more sensors include a thermometer or a temperature sensor. The temperature sensor may detect the difference in temperature on two sides of an IC or analog circuit and convert that difference to a detectable voltage or current. In devices disclosed herein, temperature sensors may measure the temperature of the inner volume of the device and/or the temperature of the environment surrounding the device, such as when the device is stored in a refrigerator or cabinet and/or when the device is opened or closed. In operation, temperature sensors for devices disclosed herein are operational and collecting temperature data while the devices is powered such that temperature data is available on-demand when a user or operator requests such information. Suitable temperature sensors for devices disclosed herein include, but are not limited to, Negative Temperature Coefficient (NTC) or Positive Temperature Coefficient (PTC) resistors, Resistance Temperature Detector (RTD) sensors, digital temperature sensors, thermocouples, and thermopiles. Temperature sensors suitable for use in devices disclosed herein generally have an operating range of −80° C. to +300° C.

In some embodiments, the one or more sensors include a pressure sensor. The pressure sensor may detect the difference in pressure on two sides of an IC or analog circuit and convert that difference to a detectable voltage or current. In devices disclosed herein, pressure sensors may measure the air pressure of the inner volume of the device and/or the pressure of the environment surrounding the device, such as when the device is stored in a refrigerator or cabinet and/or when the device is opened or closed. Alternatively, or in addition, pressure sensors may measure a change in force when a container of medicine is placed into an appropriate location within the device, such as in an insert. In operation, pressure sensors for devices disclosed herein are operational and collecting temperature data while the devices is powered such that pressure data is available on-demand when a user or operator requests such information. Suitable pressure sensors for devices disclosed herein include, but are not limited to, piezoelectric sensors, strain gauge sensors, micro-electromechanical systems (MEMS), pressure transducers, and other similar pressure sensors suitable for use with integrated circuits. Pressure sensors suitable for use in devices disclosed herein generally have an operating range of about 1 psi to about 300 psi, noting that 15 psi is approximately 1 atmosphere of pressure.

In some embodiments, the one or more sensors include a trace gas sensor. The trace gas sensor may detect a change in a gas concentration, such as the concentration of oxygen or another trace gas, and convert the measured signal to a detectable voltage or current. In devices disclosed herein, trace gas sensors may measure the concentration of one or more gases within the inner volume of the device and/or the concentration of one or more gases of the environment surrounding the device, such as when the device is stored in a refrigerator or cabinet and/or when the device is opened or closed. In particular embodiments, the trace gas sensor measures the amount of oxygen in the inner volume of the device. In operation, trace gas sensors for devices disclosed herein are operational and collecting concentration data while the devices is powered such that concentration data is available on-demand when a user or operator requests such information. Suitable trace gas sensors for devices disclosed herein include, but are not limited to, metal oxide based sensors, infrared gas sensors, catalytic diffusion sensors, variable resistance sensors, and other similar gas sensors suitable for use with integrated circuits. Trace gas sensors suitable for use in devices disclosed herein generally have an operating range of about 0 to 5 parts per million (ppm) noting that oxygen is present in ambient air at a concentration of about 400 ppm.

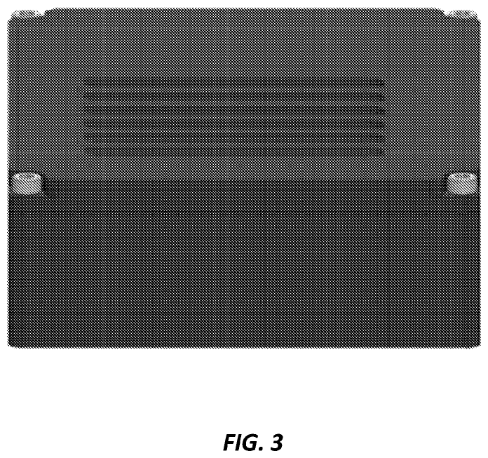

In some embodiments, the device includes an integrated communications module to permit communication between the device and a controller as described herein. For example, the sensor board may include an integrated circuit that provides for wired or wireless connectivity to the controller. Alternatively, or in addition, the device may communicate with an external communications module, such as that illustrated in FIG. 3. In this configuration, the external communications module can collect information from the device from the one or more sensors and direct the collected information to the controller. The connection between the device and the external communications module can be wired or over one or more wireless standards, e.g., Wi-Fi, BLUETOOTH®, 5G NR FR2, LTE Cat 1, LTE Cat M1 or Cat NB1 standard. In some embodiments, the external communications communication module includes both a BLUETOOTH® radio and a Wi-Fi radio. The external communications module communicates with the controller using a wireless standard as described herein.

In some embodiments, an outer surface of the device, independent of form, includes space for the application of a trademark, trade dress, or other suitable identifying indicia. Marks can be applied directly to the surfaces of the device using suitable application techniques, e.g., silk screening, adhesive stickers, and the like. In some embodiments, the device itself is manufactured by incorporating a mark therein, i.e., being shaped or colored in the fashion of the mark owner. The type of marking and branding that can be applied to the devices disclosed herein is not limited to the embodiments illustrated herein.

### Kits

The present disclosure further provides a kit including the device as described herein and one or more containers of medicine. In some embodiments, the kit includes a device as disclosed herein, a charging cable, and a receiver or docking station that connects the device to a communications module. In some embodiments, the kit includes a device as disclosed herein, a charging cable, a receiver or docking station that connects the device to a communications module, and a plurality of inserts for different shaped containers of medicines, e.g., vials, bottles, and/or injector pend. In this instance, the kit can be acquired via a retailer and be used with different types of medicines by the installation of the appropriate insert to fit the specific medicine container shape.

In some embodiments, the kit may be acquired directly from a pharmaceutical supplier and/or distributor. In this instance, the kit may include, the device as disclosed herein, a charging cable, a receiver or docking station that connects the device to a communications module, a specific insert for the medicine being used by the patient, and one or more containers of the medicine being used by the patient.

### System Description

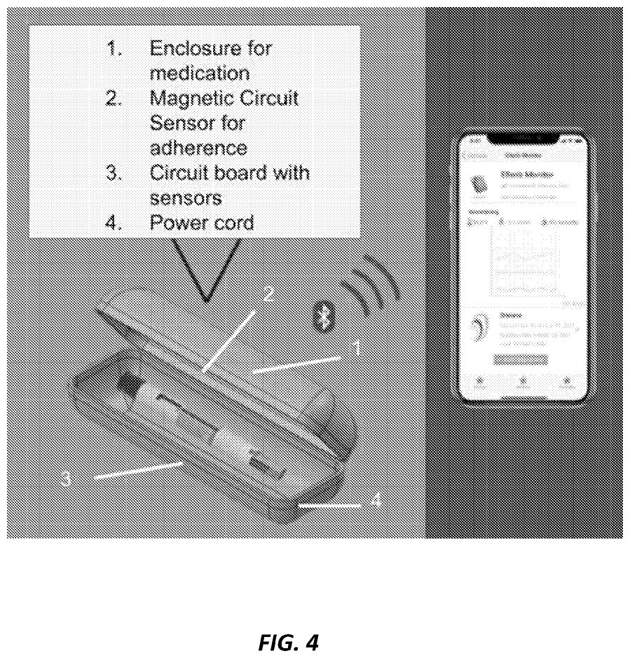

The present disclosure further provides a system for the use of the device as described herein. In some embodiments, the system is configured to alert a user about improper storage conditions or non-adherence to a medication schedule. An embodiment of a system disclosed herein, e.g., a device and a controller, is illustrated in FIG. 4. With reference to FIG. 4, the device illustrated includes a housing shown as a clamshell enclosure, a sensor board, a magnetic latch operatively connected to the sensor board, and a power connector or power cord. As illustrated, data pertaining to one or more sensors disposed on the sensor board can be transmitted to the controller, e.g., a cellular telephone, over a communications standard, e.g., BLUETOOTH® and/or WiFi. In some embodiments, the one or more sensors, e.g., thermometer, hygrometer, light meter, and pressure sensor, are configured to collect data from the storage environment. The data collected may be stored in the controller. In some embodiments, the controller can display data from the one or more sensors to a user. The user may view this data on the controller to monitor storage conditions. The type of notification may be a chime, bell, vibration, or other configuration selected by the user. A notification may be sent via a short message service (SMS) message to a user's cellular phone from the controller. In some embodiments, the controller may be in communication with a remote platform for data monitoring, storage, and processing.

The present disclosure further provides device may be reusable. In some embodiments, the system is constructed and arrange to be used with individual containers of medicine. In some embodiments, the device is constructed to be placed in a refrigerated compartment and multiple containers of medications are able to be monitored. In some embodiments, the device is a container in which one or more medication containers sit. In some embodiments, multiple devices are connected to controller for monitoring multiple medications or medication storage compartments. In some embodiments, the controller receives the data from the one or more sensors of the device and outputs collected sensor data to a user. In some embodiments, multiple devices are connected to a controller which can report data from the multiple devices to the user. In this configuration, a user can receive data and alerts about multiple medications and storage compartments at any time.

In some embodiments, a user can select one or more third party individuals or groups who are authorized to view the data and resultant alerts. In some embodiments, the user determines what information the third parties are able to view and which alerts they receive. For example, a user's physician or treatment provider may receive notifications about non-adherence to a medication schedule. A pharmacist or a person, e.g., relative or caretaker, living in the home may receive notifications about storage mismanagement. In some embodiments, the user's third parties can customize the type of notification they receive. In some embodiments, the system is used to alert the user and/or third parties of mismanagement of medication storage and lack of medication regimen compliance. In some embodiments, the system provides the user and/or third parties with proper storage protocols to follow. In some embodiments, the system provides the user and/or third party supporters with an alert if a predetermined medication schedule is not adhered to.

Many medications require storage in controlled conditions such that the chemical structure of the medication remains as intended and/or the efficacy of the medication is not impacted. Other medications may require storage in controlled conditions to prevent growth of a contaminant, such as a bacterial strain. For example, some medications, such as antibodies and biologics, antibiotics, insulin, and vaccines, generally require temperature controlled storage. In another non-limiting embodiment, some medicines are photosensitive, such as small molecule anticancer treatments, and are generally stored in low light conditions. The devices and systems disclosed herein are not limited to the type of storage conditions required for a particular medication.

The medication may be any type of medication prescribed to a subject, e.g., a human subject. In some embodiments, the medication to be stored is an antibiotic, antiviral, anti-inflammatory, antidepressant, antacid, opioid, steroid, analgesic, anticonvulsant, antihistamine, antihypertensive, antifungal, antiemetic, antipyretic, cough suppressant, cytotoxic, decongestant, antidiarrheal, hormone, laxative, hypoglycemic, immunosuppressant, muscle relaxant, sedative, statin, tranquilizer, or vitamin. The medication may impact any cell, tissue, organ, of the body, including the skin, heart, eye, stomach, intestine, liver, spleen, pancreas, heart, brain, lung, nerves, mouth, teeth, muscle, bone, ear, hair, bladder, kidney, ovary, uterus, testes, prostate, connective tissue, cartilage. The medication may act on any system or function of the body, including the digestive system, cardiovascular system, immune system, nervous system, respiratory system, endocrine system, reproductive system, or musculoskeletal system. The medication may be a small molecule drug or biologic drug, e.g., a nucleic acid, peptide, protein, or oligosaccharide, or a combination thereof.

In some embodiments, the medication is an analgesic. For example, the medication may be ibuprofen, acetaminophen, aspirin, or naproxen. In some embodiments, the medication is an antibiotic, such as amoxicillin, amoxicillin and clavulanate, ampicillin, dicloxacillin, oxacillin, penicillin V potassium, demeclocycline, doxycycline, eravacycline, minocycline, omadacycline, sarecycline, tetracycline, cefaclor, cefadroxil, cefdinir, cephalexin, cefprozil, cefdinir, cefepime, cefiderocol, cefotaxime, cefotetan, ceftaroline, ceftazidime, ceftriaxone, cefuroxime, ciprofloxacin, delafloxacin, levofloxacin, moxifloxacin, gemifloxacin, clindamycin, lincomycin, azithromycin, clarithromycin, erythromycin, fidaxomicin, sulfamethoxazole and trimethoprim, sulfasalazine, dalbavancin, oritavancin, telavancin, vancomycin, gentamicin, tobramycin, amikacin, imipenem and cilastatin, meropenem, ertapenem, bacitracin, neomycin, and polymyxin B.

In some embodiments, the medication is a hormone or hormone suppressor, such as 5-alpha-reductase inhibitors, adrenal cortical steroids, e.g., corticotropin, glucocorticoids, or mineralocorticoids, adrenal corticosteroid inhibitors, antiandrogens, antidiuretic hormones, antigonadotropic agents, antithyroid agents, aromatase inhibitors, calcimimetics, calcitonin, estrogen receptor antagonists, gonadotropin-releasing hormone antagonists, growth hormone receptor blockers, growth hormones, insulin, insulin-like growth factors, melanocortin receptor agonists, miscellaneous hormones, parathyroid, hormone and analogs, progesterone receptor modulators, prolactin inhibitors, selective estrogen receptor modulators, sex hormones, e.g., androgens and anabolic steroids, contraceptives estrogens, gonadotropin releasing hormones, gonadotropins, progestins, or combinations thereof, somatostatin and somatostatin analogs, synthetic ovulation stimulants, and thyroid drugs.

In some embodiments, the medication to be stored and monitored is an antibody or an antibody fragment, e.g., a bispecific fragment, a trispecific fragment, Fab, F(ab′)2, or a single-chain variable fragment (scFv). In some embodiments, the antibody or fragment is 3F8,

Abagovomab, Abciximab, Abituzumab, Abrilumab, Acritumomab, Actoxumab, Adalimumab, Adalimumab-atto, Adecatumumab, Ado-trastuzumab emtansine, Aducanumab, Afasevikumab, Afelimomab, Afutuzumab, Alacizumab pegol, ALD518, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, Crenezumab, Crotedumab, CR6261, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab,
Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Inebilizumab, Infliximab, Infliximab-dyyb, Intetumumab, Inolimomab, Inotuzumab ozogamicin, Ipilimumab, Iratumumab, Isatuximab, Itolizumab, Ixekizumab, Keliximab, Labetuzumab, Lambrolizumab, Lampalizumab, Lanadelumab, Landogrozumab, Laprituximab emtansine, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Mapatumumab, Margetuximab, Maslimomab, Mavrilimumab, Matuzumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tildrakizumab, Tigatuzumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, Zolimomab aritox, 8H9, Abrezekimab, Aprutumab ixadotin, Atidortoxumab, Azintuxizumab vedotin, Belantamab mafodotin, BCD-100, Berlimatoxumab, Bersanlimab, Birtamimab, BIVV009, BMS-98601 6, Cetrelimab, Cibisatamab, Cosfroviximab, Cusatuzumab, Dezamizumab, DS-8201, Enapotamab vedotin, Etigilimab, Faricimab, FBTA05, Gancotamab, Gosuranemab, GSK2831781, lanalumab, IBI308, Ifabotuzumab, lladatuzumab vedotin, Imaprelimab, Infliximab-abda, Infliximab-qbtx, lomab-B, Iscalimab, Istiratumab, Lacnotuzumab, Ladiratuzumab vedotin, Larcaviximab, Lenvervimab, Leronlimab, Lesofavumab, Letolizumab, Loncastuximab tesirine, Losatuxizumab vedotin, Lupartumab amadotin, Lutikizumab, MABpl, Marstacimab, Modotuximab, Mosunetuzumab, Naxitamab, NEOD001, Netakimab, Netakimab, Oleclumab, Olendalizumab, OMS721, Onvatilimab, Otilimab, PDR001, Ravagalimab, Ravulizumab, Remtolumab, Romilkimab, Rozanolixizumab, SA237, Samrotamab vedotin, Setrusumab, SHP647, Sirtratumab vedotin, Spartalizumab, Suptavumab, Sutimlimab, Suvratoxumab, Talacotuzumab, Tavolimab, Telisotuzumab vedotin, Tepoditamab, Tibulizumab, Timigutuzumab, Tiragotumab, Tislelizumab, Tomuzotuximab, TRBS07, Vanalimab, Varisacumab, Vonlerolizumab, Vopratelimab, Vunakizumab, XMAB-5574, Zenocutuzumab, or Zolbetuximab.

In some embodiments, the antibody or fragment is 8H9, Abrezekimab, Aprutumab ixadotin, Atidortoxumab, Azintuxizumab vedotin, Belantamab mafodotin, BCD-100, Berlimatoxumab, Bersanlimab, Birtamimab, BIVV009, BMS-986016, Cetrelimab, Cibisatamab, Cosfroviximab, Cusatuzumab, Dezamizumab, DS-8201, Enapotamab vedotin, Etigilimab, Faricimab, FBTA05, Gancotamab, Gosuranemab, GSK2831781, lanalumab, IBI308, Ifabotuzumab, lladatuzumab vedotin, Imaprelimab, Infliximab-abda, Infliximab-qbtx, lomab-B, Iscalimab, Istiratumab, Lacnotuzumab, Ladiratuzumab vedotin, Larcaviximab, Lenvervimab, Leronlimab, Lesofavumab, Letolizumab, Loncastuximab tesirine, Losatuxizumab vedotin, Lupartumab amadotin, Lutikizumab, MABpl, Marstacimab, Modotuximab, Mosunetuzumab, Naxitamab, NEOD001, Netakimab, Netakimab, Oleclumab, Olendalizumab, OMS721, Onvatilimab, Otilimab, PDR001, Ravagalimab, Ravulizumab, Remtolumab, Romilkimab,

Rozanolixizumab, SA237, Samrotamab vedotin, Setrusumab, SHP647, Sirtratumab vedotin, Spartalizumab, Suptavumab, Sutimlimab, Suvratoxumab, Talacotuzumab, Tavolimab, Telisotuzumab vedotin, Tepoditamab, Tibulizumab, Timigutuzumab, Tiragotumab, Tislelizumab, Tomuzotuximab, TRBS07, Vanalimab, Varisacumab, Vonlerolizumab, Vopratelimab, Vunakizumab, XMAB-5574, Zenocutuzumab, or Zolbetuximab.

In some embodiments, the medication to be stored is ledipasvir/sofosbuvir, insulin glargine, lenalidomide, pneumococcal 13-valent conjugate vaccine, fluticasone/salmeterol, elvitegravir/cobicistat/emtricitabine/tenofovir alafenamide, emtricitabine, rilpivirine and tenofovir alafenamide, emtricitabine/tenofovir alafenamide, grazoprevir/elbasvir, coagulation factor VIla recombinant, epoetin alpha, Aflibercept or etanercept.

In some embodiments, the medication to be stored is Abatacept, AbobotulinumtoxinA, Agalsidase beta, Albiglutide, Aldesleukin, Alglucosidase alpha, Alteplase (cathflo activase), Anakinra, Asfotase alpha, Asparaginase, Asparaginase Erwinia chrysanthemi, Becaplermin, Belatacept, Collagenase, Collagenase clostridium histolyticum, Darbepoetin alpha, Denileukin diftitox, Dornase alpha, Dulaglutide, Ecallantide, Elosulfase alpha, Etanercept-szzs, Filgrastim, Filgrastim-sndz, Galsulfase, Glucarpidase, Idursulfase, IncobotulinumtoxinA, Interferon alpha-2b, Interferon alpha-n3, Interferon beta-1a, Interferon beta-1b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alpha-2a, Peginterferon alpha-2a co-packaged with ribavirin, Peginterferon alpha-2b, Peginterferon beta-la, Pegloticase, Rasburicase, Reteplase, Rilonacept, RimabotulinumtoxinB, Romiplostim, Sargramostim, Sebelipase alpha, Tbo-filgrastim, Tenecteplase, or Ziv-aflibercept.

In some embodiments, the diagnostic agent is tuberculin purified protein derivative, hyrotropin alpha, secretin, soluble transferrin receptor, troponin, B-type natriuretic peptide, iobenguane 1123, florbetapir F18, perflutren, gadoterate meglumine, florbetaben F18, flutemetamol F18, gadoterate meglumine, isosulfan blue, regadenoson, technetium Tc99, tilmanocept, and florbeta.

In some embodiments, the medication to be stored is Amikacin, Gentamicin, Kanamycin, Neomycin, Netilmicin, Tobramycin, Paromomycin, Streptomycin, Spectinomycin, Ansamycins, Geldanamycin, Herbimycin, Rifaximin, Carbacephem, Loracarbef, Carbapenems, Ertapenem, Doripenem, Imipenem/Cilastatin, Meropenem, Cephalosporins, Cefadroxil, Cefazolin, Cephradine, Cephapirin, Cephalothin, Cefalexin, Cefaclor, Cefoxitin, Cefotetan, Cefamandole,

Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, Ceftobiprole, Glycopeptides, Teicoplanin, Vancomycin, Telavancin, Dalbavancin, Oritavancin, Lincosamides, Clindamycin, Lincomycin, Lipopeptide, Daptomycin, Macrolides, Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin, Oxazolidinones, Linezolid, Posizolid, Radezolid, Torezolid, Penicillins, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Penicillin combinations, moxicillin/clavulanate, Ampicillin/sulbactam, Piperacillin/tazobactam, Ticarcillin/clavulanate, Polypeptides, Bacitracin, Colistin, Polymyxin B, Quinolones/Fluoroquinolones, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Sulfonamides, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), Sulfonamidochrysoidine, Tetracyclines, Demeclocycline, Doxycycline, Metacycline, Minocycline, Oxytetracycline, Drugs against mycobacteria, Clofazimine, Dapsone, Capreomycin, Cycloserine, Ethambutol, Ethionamide, Isoniazid, Pyrazinamide, Rifampicin, Rifabutin, Rifapentine, Streptomycin, Others, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, and Trimethoprim.

In some embodiments, the medication to be stored is an immunotherapy. In some embodiments, the immunotherapy is a PD-1 inhibitor such as a PD-1 antibody, a PD-L1 inhibitor such as a PD-L1 antibody, a CTLA-4 inhibitor such as a CTLA-4 antibody, a CSF-1 R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor. In some embodiments, the immunotherapy is a PD-1 inhibitor (e.g., nivolumab, pembrolizumab, pidilizumab, BMS 936559, and MPDL3280A). In some embodiments, the immunotherapy is a PD-L1 inhibitor (e.g., atezolizumab and MED14736). In some embodiments, the immunotherapy is a CTLA-4 inhibitor (e.g., ipilimumab). In some embodiments, the immunotherapy is a CSF-1 R inhibitor (e.g., pexidartinib and AZD6495). In some embodiments, the immunotherapy is an IDO inhibitor (e.g., norharmane, rosmarinic acid, and alpha-methyl-tryptophan). In some embodiments, the immunotherapy is an Al adenosine inhibitor (e.g., 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, or N-0861). In some embodiments, the immunotherapy is an A2A adenosine inhibitor (e.g., ATL-4444, istradefylline, MSX-3, preladenant, SCH-58261, SCH-412,348, SCH-442,416, ST-1535, VER-6623, VER-6947, VER-7835, viadenant, or ZM-241,385). In some embodiments, the immunotherapy is an A2B adenosine inhibitor (e.g., ATL-801, CVT-6883, MRS-1706, MRS-1754, OSIP-339,391, PSB-603, PSB-0788, or PSB-1115). In some embodiments, the immunotherapy is an A3A adenosine inhibitor (e.g., KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421). In some embodiments, the immunotherapy is an arginase inhibitor (e.g., an arginase antibody, (2S)-(+)-amino-5-iodoacetamidopentanoic acid, NG-hydroxy-L-arginine, (2S)-(+)-amino-6-iodoacetamidohexanoic acid, or (R)-2-amino-6-borono-2-(2-(piperidin-1-yl)ethyl)hexanoic acid. In some embodiments, the immunotherapy is an HDAC inhibitor (e.g., valproic acid, SAHA, or romidepsin). In some embodiments, the immunotherapy is a toll-like receptor activator. In some embodiments, the immunotherapy is a RIG-I-like receptor activator. In some cases, the immunotherapy is a stimulator of interferon genes (STING) pathway activator. In some embodiments, the immunotherapy is an Interleukin-1 receptor agonist, e.g., an IL-R1 antagonist. In some embodiments, the immunotherapy is a PTEN inhibitor, e.g., a bisperoxovanadium compound. In some embodiments, the immunotherapy is a tumor necrosis factor receptor (TNFR), e.g., TNFR-1 or TNFR-2 inhibitor. In some embodiments, the immunotherapy is a Lymphocyte-activation gene 3 (LAG-3) inhibitor, e.g., GSK2831781.

In some embodiments, the medication to be stored is ledipasvir/sofosbuvir, insulin glargine, lenalidomide, pneumococcal 13-valent conjugate vaccine, fluticasone/salmeterol, elvitegravir/cobicistat/emtricitabine/tenofovir alafenamide, emtricitabine, rilpivirine and tenofovir alafenamide, emtricitabine/tenofovir alafenamide, grazoprevir/elbasvir, coagulation factor VIIa recombinant, epoetin alpha, Aflibercept or etanercept.

In some embodiments, the medicine to be stored is Abatacept, Abobotulinumtoxin A, Agalsidase beta, Albiglutide, Aldesleukin, Alglucosidase alpha, Alteplase (cathflo activase), Anakinra, Asfotase alpha, Asparaginase, Asparaginase erwinia chrysanthemi, Becaplermin, Belatacept, Collagenase, Collagenase clostridium histolyticum, Darbepoetin alpha, Denileukin diftitox, Dornase alpha, Dulaglutide, Ecallantide, Elosulfase alpha, Etanercept-szzs, Filgrastim, Filgrastim-sndz, Galsulfase, Glucarpidase, Idursulfase, IncobotulinumtoxinA, Interferon alpha-2b, Interferon alpha-n3, Interferon beta-1a, Interferon bete-1b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alpha-2a, Peginterferon alpha-2a co-packaged with ribavirin, Peginterferon alpha-2b, Peginterferon beta-1a, Pegloticase, Rasburicase, Reteplase, Rilonacept, RimabotulinumtoxinB, Romiplostim, Sargramostim, Sebelipase alpha, Tbo-filgrastim, Tenecteplase, or Ziv-aflibercept.

In some embodiments, the medication to be stored is tuberculin purified protein derivative, hyrotropin alpha, secretin, soluble transferrin receptor, troponin, B-type natriuretic peptide, iobenguane I 123, florbetapir F 18, perflutren, gadoterate meglumine, florbetaben F 18, flutemetamol F 18, gadoterate meglumine, isosulfan blue, regadenoson, technetium Tc 99m tilmanocept, florbetaben F 18, perflutren, regadenoson, or flutemetamol F 18.

The medication to be stored by in liquid or solid form. In some embodiments, the medication is a syrup, mixture, drop, solution, or gel. In other embodiments, the medication is a tablet, capsule, cream, or powder. The medication may be provided to contain a single dosage or a plurality of dosages, e.g., 2, 3, 4, 5, 6, 7, 8, 9, 10, 15, 20, 25, 30, 40, 50, or more dosages. The medication may be provided in individual dosage forms, for example, tablets for consumption, or may require the user to remove a portion of the medication at each dosage for consumption. The medication may be disposed in a delivery system, such as in a syringe, injector pen, implant, patch, or inhaler. The medication may be administered in any route, including oral, subcutaneous, or topical administration. In certain embodiments, the medicine is provided in an injector pen. For example, the injector pen can be filled with a hormone, such as epinephrine, i.e., the injector pen in an EPIPEN®. In other non-limiting embodiments, the injector pen may be filled with insulin. Other medicines suitable for subcutaneous injection using an injector pen are within the scope of this disclosure.

In an embodiment, the medication is housed in a cylinder-shaped container. In an embodiment, the medication is housed in a pen-shaped container. In an embodiment, the medication is housed in a vial. In an embodiment, the medication is housed in a syringe. In an embodiment, the medication is housed in an inhaler.

### Methods of Use

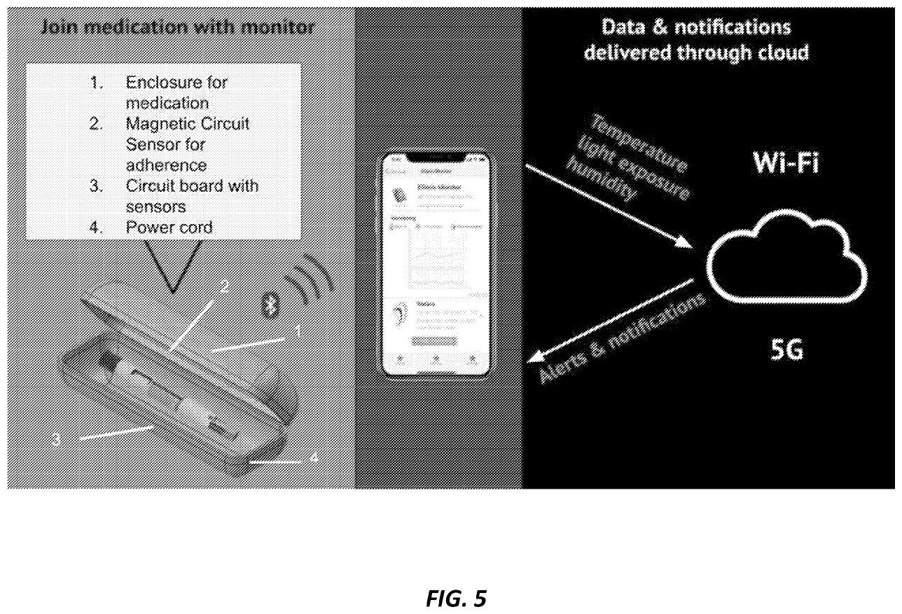

The present disclosure further provides methods of use of the device embodiments and systems as described herein. In some embodiments, the one or more sensors, e.g., thermometer, light meter, hygrometer, and pressure-weight sensor, are configured to collect data about storage conditions of medications. In some embodiments, controller is located outside of the medication storage area. In some embodiments, said controller receives sensor data. FIG. 5 illustrates a method of managing the use of a medication using a system, e.g., a device and a controller, as disclosed herein. In operation, the device is opened, upon which the sensor positioned at the latch or clasp of the device communicates to the controller that the device has been opened. The one or more sensors of the sensor board, e.g., light intensity, temperature, pressure, and/or humidity, detect environmental changes, e.g., going from a dark environment to a lit environment, and the device communicated data pertaining to these variables to the controller over a suitable communications standard, e.g., BLUETOOTH®, WiFi, or the like. The collected data can be directed to a cloud-based storage system or server for processing into information pertaining to the medication compliance schedule for the user. The cloud-based storage or server can direct one or more alerts regarding medication compliance to the controller. In some embodiments, the controller then converts these data inputs into a user-readable format. In some embodiments, the user sets the storage parameters and/or medication schedule by manually adding the information into a user interface of the controller. In some embodiments, said inputs are relayed to the controller, which compares the sensor data to the parameter inputs and sends an alert if the inputs are not within the parameters.

The present disclosure further provides for a method of use where the pressure/weight sensor may be utilized to determine user adherence to a medication schedule. In some embodiments, the pressure/weight sensor checks for a weight difference in the medication bottle before and after a predetermined medication-consumption time. In case of a user deviating from a predetermined medication schedule or taking too much of a medication, the system may create an alert for the user and/or third parties. In some embodiments, the user selects which types of notifications to send to one or more third parties. For example, a user may choose to send a medical provider only notifications about medication regimen adherence, or both adherence and medication storage mismanagement alerts. A user may choose to include pharmacists or family members as additional third parties to whom notifications are sent. In some embodiments, the third party supporters can control which notifications they receive. The hub device may send a report at regular intervals to the user or the user's application. In some embodiments, a user selects a one or more third party supporters to whom a report is also sent. In some embodiments, the controller retains the measured sensor data and deliver a notification to a user if the measured sensor data is outside a set parameter.

### ENUMERATED EMBODIMENTS

1. A medication management device comprising,
   a housing constructed and arranged to receive a container of medication;
   one or more sensors; and
   a power source, wherein the medication management device is capable of monitoring one or more environmental parameters.
2. The device of embodiment 1, wherein the one or more environmental parameters comprises temperature, light, pressure, relative humidity, oxygen content, or a combination thereof.
3. The device of any one of the preceding embodiments, wherein the one or more environmental parameters comprises temperature, light, pressure, relative humidity, or a combination thereof.
4. The device of any one of the preceding embodiments, wherein the one or more environmental parameters comprises temperature, light, relative humidity, or a combination thereof.
5. The device of any one of the preceding embodiments, wherein the one or more sensors comprises a thermometer, a hygrometer, a light sensor, a pressure sensor, or a combination thereof.
6. The device of any one of the preceding embodiments, wherein the power source is a battery, e.g., a rechargeable battery.
7. The device of any one of the preceding embodiments, wherein the power source comprises a connection to an electrical receptacle.
8. The device of any one of the preceding embodiments, wherein the power source can be powered wirelessly, e.g., a wireless charging coil, e.g., a Qi coil.
9. The device of any one of the preceding embodiments, constructed and arranged to be releasably attached to a container of medication.
10. The device of any one of the preceding embodiments, constructed and arranged to substantially enclose one or more containers of medication.
11. The device of any one of the preceding embodiments, further comprising a communication module configured to collect and transmit data from the one or more sensors.
12. The device of any one of the preceding embodiments, wherein the one or more sensors are disposed on a lower portion of the device.
13. The device of any one of the preceding embodiments, wherein the one or more sensors are disposed on a side wall of the device.
14. A medication management device comprising,
   a housing constructed and arranged to receive a container of medication;
   one or more sensors;
   a communication module; and
   a power source, wherein the medication management device is capable of monitoring one or more environmental parameters.
15. The device of embodiment 14, wherein the communication module comprises a wireless communications module.
16. The device of any one of the preceding embodiments, wherein the communication module comprises a BLUETOOTH® radio.
17. The device of any one of the preceding embodiments, wherein the housing further comprises a securing mechanism.
18. The device of any one of the preceding embodiments, wherein the housing further comprises a securing mechanism comprising a magnetic switch operatively coupled to a sensor board of the device.
19. The device of any one of the preceding embodiments, adapted to hold an injector pen medication delivery system.
20. The device of any one of the preceding embodiments, adapted to hold an injector pen medication delivery system comprising a hormone pen.
21. The device of any one of the preceding embodiments, adapted to hold an injector pen medication delivery system comprising an epinephrine injector pen, e.g., an EPIPEN®, or an insulin injector pen.
22. The device of any one of the preceding embodiments, further comprising an insert dimensioned to fit within the housing constructed and arranged to hold the container of medicine.
23. The device of any one of the preceding embodiments, further comprising an insert comprising a hard polymer dimensioned to fit within the housing constructed and arranged to hold the container of medicine.
24. The device of any one of the preceding embodiments, further comprising an insert comprising a soft polymer, e.g., urethane foam, i.e., memory foam, dimensioned to fit within the housing constructed and arranged to hold the container of medicine.
25. The device of any one of embodiments 22-24, wherein the insert is shaped to fit a standard injector pen medication delivery system.
26. The device of any one of embodiments 22-24, wherein the insert is shaped to fit one or more vials of a medicine.
27. The device any one of the preceding embodiments, having a length of about 25 mm to about 300 mm.
28. The device any one of the preceding embodiments, having a width of about 25 mm to about 300 mm.
29. The device of any one of the preceding embodiments, having a thickness or height of about 25 mm to about 300 mm.
30. The device of any one of the preceding embodiments, having a length of about 200 mm, a width of about 64 mm, and a height or thickness of about 64 mm.
31. The device of any one of the preceding embodiments, constructed and arranged as an open container.
32. The device of any one of the preceding embodiments, constructed and arranged as a clamshell comprising a first half and a second half connected by a hinge.
33. The device of embodiment 32, wherein one or both halves of the clamshell are made from a same material.
34. The device of embodiment 32, wherein each half of the clamshell are made from different material.
35. The device of any one of embodiments 32-34, wherein one or both halves of the clamshell are made from one or more of a metal, e.g., steel, brass, bronze, aluminum, or zinc, a finished wood, e.g., alder, balsa, beech, hickory, mahogany, maple, oak, teak, walnut, bamboo, cedar, Douglas fir, juniper, pine, redwood, spruce, and yew, or a polymer, e.g., polyethylene terephthalate (PETE or PET), polyethylene, polyvinyl chloride (PVC), polypropylene, polystyrene, polylactic acid, polycarbonate, acrylic, acetal, polyether ether ketone (PEEK), polyoxymethylene, nylon, acrylonitrile butadiene styrene (ABS), and other common polymers and blends thereof.
36. The device of any one of the preceding embodiments, wherein an inner surface of the device comprises a surface coating.
37. The device of any one of the preceding embodiments, wherein an inner surface of the device comprises a surface coating comprising an absorbent coating.
38. The device of any one of the preceding embodiments, wherein an inner surface of the device comprises a surface coating comprising a flocked coating.
39. The device of any one of the preceding embodiments, wherein an inner surface of the device comprises a surface coating comprising a fabric liner, e.g., microfiber or microsuede.
40. The device of any one of the preceding embodiments, wherein an inner surface of the device comprises a surface coating comprising a moisture or stain repellant coating, e.g., TEFLON® and SCOTCHGUARD®.
41. A medication management system, comprising:
   a medication management device comprising,
      a housing constructed and arranged to receive a container of medication;
      one or more sensors; and
      a power source, wherein the medication management device is capable of monitoring one or more environmental parameters; and
   a controller operatively connected to the one or more sensors.
42. The system of embodiment 41, wherein the controller comprises a non-transitory computer-readable medium storing instructions which, when executed by the controller, provide an interface configured to display data pertaining to measurements from the one or more sensors.
43. The system of any one of the preceding embodiments, wherein the medication management device and the controller communicate wirelessly.
44. The system of any one of the preceding embodiments, wherein the medication management device and the controller communicate over a wired connection.
45. The system of any one of the preceding embodiments, wherein the controller is configured with a dosing schedule for a medication.
46. The system of any one of the preceding embodiments, wherein the controller is configured to provide an alert to a user upon detecting a deviation from the medication dosing schedule.
47. The system of any one of the preceding embodiments, wherein the controller is configured to provide an alert to a user based on a measurement from the one or more sensors exceeding a predetermined threshold.
48. The system of any one of the preceding embodiments, wherein the controller is configured to permit access to data pertaining to measurement from the one or more sensors and/or deviations from the medication dosing schedule by one or more third parties.
49. The system of any one of the preceding embodiments, wherein the controller is configured to provide an alert to the one or more third parties upon detecting a deviation from the medication dosing schedule or based on a measurement from the one or more sensors exceeding a predetermined threshold.
50. The system of any one of the preceding embodiments, further comprising an external communication module operatively coupled to one or both of the medication management device and the controller.
51. The system of any one of the preceding embodiments, further comprising an external communication module comprising a wireless communication module operatively coupled to one or both of the medication management device and the controller.
52. The system of any one of the preceding embodiments, further comprising an external communication module comprising a wireless communication module comprising a WiFi radio operatively coupled to one or both of the medication management device and the controller.
53. The system of any one of the preceding embodiments, further comprising an external communication module comprising a wireless communication module comprising a WiFi radio and a BLUETOOTH® radio operatively coupled to one or both of the medication management device and the controller.
54. A method of managing the use of a medication comprising:
   providing a medication management system comprising a medication management device comprising one or more sensors and a controller comprising a user interface,
   interfacing one or more containers of medication within the medication management device; and
   monitoring one or more environmental parameters of the medication management device.
55. The method of embodiment 54, further comprising configuring the controller with a dosing schedule for the medication.
56. The method of any one of the preceding embodiments, further comprising configuring the controller with predetermined threshold values for measurements from the one or more sensors.
57. The method of any one of the preceding embodiments, comprising providing an alert to a user upon detection of a deviation from the medication dosing schedule.
58. The method of any one of the preceding embodiments, comprising providing an alert to a user based on a measurement from the one or more sensors exceeding the predetermined threshold.
59. The method of any one of the preceding embodiments, wherein the user interface of the controller displays information pertaining to the deviation from the medication dosing schedule or measurement from the one or more sensors exceeding the predetermined threshold to the user.
60. The method of any one of the preceding embodiments, further comprising providing an alert to one or more third parties based on a measurement from the one or more sensors exceeding the predetermined threshold.
61. The method of any one of embodiments 54-60, further comprising providing an alert to one or more third parties based on upon detection of a deviation from the medication dosing schedule.
62. The method of any one of the preceding embodiments, wherein the medication management device is connectable to an individual container of medication.
63. The method of any one of the preceding embodiments, wherein the medication management system is constructed and arranged to store one or more containers of medication.

### EXAMPLES

The function and advantages of these and other embodiments can be better understood from the following examples. These examples are intended to be illustrative in nature and are not considered to be in any way limiting the scope of the invention.

### Example 1

### Assembly of Device and Exemplary Use

A device was assembled according the drawing provided in FIG. 1. In particular, a circular piece of a polymer fitted with a rechargeable battery and a sensor board as illustrated in FIG. 2 comprising a weight sensor. This device was enclosed within a polymer enclosure leaving open the weight sensor's active area. In operation, the weight sensor was first calibrated without any containers of medicine on the active area to provide for a zero-mass measure. A container of medicine containing a dosing regimen was placed onto the weight sensor and a weight collected to determine the combined weight of the medicine on the container and the container. The device was tested for its ability to measure the changes in mass of the container of medicine as the medicine was used by a patient by removing the container from the device, removing a dose of medicine, and placing the container back onto the device.

### Example 2

### Assembly of Device and Exemplary Use

A device is to be assembled according the drawing provided in FIG. 4. In particular, the device is to be fabricated in a clamshell design made from an impact-resistant polymer with the two halves of the device connected by a hidden hinge akin to an eyeglasses case. Inside the device, both halves will be lined with a soft material such as a felt liner or a flocked liner. One half of the device will be fitted with a rechargeable battery and a sensor board as illustrated in FIG. 2 comprising temperature, pressure, and light sensors. These sensors will be used to measure the amount of light that enters the device when opened, the temperature inside of the device, and the pressure applied by a container of medicine in the device. As illustrated, the device is configured to store a container of medicine in the form of an injector pen, such as an insulin pen, an injector pen containing a biologic drug, or a hormone injector pen. In operation, the injector pen is to be placed into the lower half of the device and disposed over the sensor board. When the top half of the device is closed, the magnetic circuit that is to be incorporated into the closure system of the device completes a circuit when the device is closed. When the device is subsequently opened, the contacts of the magnetic switch are opened, creating a gap in the magnetic switch reeds. The device is to include a removable insert made from a shape memory material that sits in the lower half of the device and includes an injector pen-shaped cutout where a user is to place the injector pen once a medication dose has been delivered. The insert aids in proper placement of the injector pen over the sensor board of the device for accurate measurements by the pressure sensor. As further illustrated, the device includes a power cord that is to be used to charge the battery of the device, e.g., over USB, or otherwise provide power to the device when energized by a household receptable.

This device is to be tested for its ability to maintain an exemplary medication in an injector pen delivery system at a certain temperature, light flux exposure, and pressure over 48 hours.

### Example 3

### Assembly of Device and Exemplary Use

A device is to be assembled according the drawing provided in FIGS. 4 and 5. In particular, the device is to be fabricated in a clamshell design made from an impact-resistant polymer with the two halves of the device connected by a hidden hinge akin to an eyeglasses case. Inside the device, both halves will be lined with a soft material such as a felt liner or a flocked liner. One half of the device will be fitted with a rechargeable battery and a sensor board as illustrated in FIG. 2 comprising temperature, pressure, and light sensors. These sensors will be used to measure the amount of light that enters the device when opened, the temperature inside of the device, and the pressure applied by a container of medicine in the device. The device further is to include a communications module including a wireless BLUETOOTH® radio that is to be configured to communicate data from the sensor board pertaining to temperature, pressure, and light flux to a communications platform, shown as a cellular phone, that has been paired with the device. The cellular phone is to include a companion web-based application that collects the data from the sensor board and provides a readout or display to a user or third party, such as a medical provider. As illustrated in FIG. 5, the data from the companion web-based application is to be delivered to cloud based storage from the communications platform over an applicable data communications standard, e.g., Wi-Fi, 5G NR FR2, LTE Cat 1, LTE Cat M1 or Cat NB1. The cloud based storage system is to be configured to interpret the data from the companion web-based application and provide alerts and notifications back to the companion web-based application for the user or third party to review.

As illustrated, the device is configured to store a container of medicine in the form of an injector pen, such as an insulin pen, an injector pen containing a biologic drug, or a hormone injector pen, e.g., an insulin pen or an epinephrine pen, i.e., an EPIPEN®. In operation, the injector pen is to be placed into the lower half of the device and disposed over the sensor board. When the top half of the device is closed, the magnetic circuit that is to be incorporated into the closure system of the device completes a circuit when the device is closed. When the device is subsequently opened, the contacts of the magnetic switch are opened, creating a gap in the magnetic switch reeds. The device is to include a removable insert made from a shape memory material that sits in the lower half of the device and includes an injector pen-shaped cutout where a user is to place the injector pen once a medication dose has been delivered. The insert aids in proper placement of the injector pen over the sensor board of the device for accurate measurements by the pressure sensor. As further illustrated, the device includes a power cord that is to be used to charge the battery of the device, e.g., over USB, or otherwise provide power to the device when energized by a household receptable.

This device is to be tested for its ability to maintain an exemplary medication in an injector pen delivery system at a certain temperature, light flux exposure, and pressure over 48 hours.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only. Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. A medication management device comprising,
a housing constructed and arranged to receive a container of medication;
one or more sensors; and
a power source, wherein the medication management device is capable of monitoring one or more environmental parameters.

2. The device of claim 1, wherein the one or more environmental parameters comprises temperature, light, pressure, relative humidity, oxygen content, or a combination thereof.

3. The device of claim 1, wherein the one or more environmental parameters comprises temperature, light, pressure, relative humidity, or a combination thereof.

4. The device of claim 1, wherein the one or more environmental parameters comprises temperature, light, relative humidity, or a combination thereof.

5. The device of claim 1, wherein the one or more sensors comprises a thermometer, a hygrometer, a light sensor, a pressure sensor, or a combination thereof.

6. The device of claim 1, wherein the power source is a battery, e.g., a rechargeable battery.

7. The device of claim 1, wherein the power source comprises a connection to an electrical receptacle.

8. The device of claim 1, constructed and arranged to be releasably attached to a container of medication.

9. The device of claim 1, constructed and arranged to substantially enclose one or more containers of medication.

10. The device of claim 1, further comprising a communication module configured to collect and transmit data from the one or more sensors.

11. The device of claim 1, wherein the one or more sensors are disposed on a lower portion of the device.

12. The device of claim 1, wherein the one or more sensors are disposed on a side wall of the device.

13. A medication management device comprising,
a housing constructed and arranged to receive a container of medication;
one or more sensors;
a communication module; and
a power source, wherein the medication management device is capable of monitoring one or more environmental parameters.

14. The device of claim 13, wherein the communication module comprises a wireless communications module.

15. The device of claim 14, wherein the wireless communication module comprises a BLUETOOTH® radio.

16. The device of any one of claims 1-15, wherein the housing further comprises a securing mechanism.

17. The device of claim 16, wherein the securing mechanism comprises a magnetic switch operatively coupled to a sensor board of the device.

18. The device of any one of claims 1-17, adapted to hold an injector pen medication delivery system.

19. The device of claim 18, wherein the injector pen is a hormone pen.

20. The device of claim 19, wherein the hormone injector pen is an epinephrine injector pen or an insulin injector pen.

21. The device of any one of claims 1-20, further comprising an insert dimensioned to fit within the housing constructed and arranged to hold the container of medicine.

22. The device of any one of claims 1-22, having a length of about 25 mm to about 300 mm.

23. The device of any one of claims 1-22, having a width of about 25 mm to about 300 mm.

24. The device of any one of claims 1-23, having a thickness or height of about 25 mm to about 300 mm.

25. The device of any one of claims 1-24, having a length of about 200 mm, a width of about 64 mm, and a height or thickness of about 64 mm.

26. A medication management system, comprising:
a medication management device comprising,
a housing constructed and arranged to receive a container of medication;
one or more sensors; and
a power source, wherein the medication management device is capable of monitoring one or more environmental parameters; and
a controller operatively connected to the one or more sensors.

27. The system of claim 26, wherein the controller comprises a non-transitory computer-readable medium storing instructions which, when executed by the controller, provide an interface configured to display data pertaining to measurements from the one or more sensors.

28. The system of claim 26, wherein the medication management device and the controller communicate wirelessly.

29. The system of claim 26, wherein the medication management device and the controller communicate over a wired connection.

30. The system of claim 26, wherein the controller is configured with a dosing schedule for a medication.

31. The system of claim 22, wherein the controller is configured to provide an alert to a user upon detecting a deviation from the medication dosing schedule.

32. The system of claim 26, wherein the controller is configured to provide an alert to a user based on a measurement from the one or more sensors exceeding a predetermined threshold.

33. The system of claim 26, wherein the controller is configured to permit access to data pertaining to measurement from the one or more sensors and/or deviations from the medication dosing schedule by one or more third parties.

34. The system of claim 33, wherein the controller is configured to provide an alert to the one or more third parties upon detecting a deviation from the medication dosing schedule or based on a measurement from the one or more sensors exceeding a predetermined threshold.

35. The system of any one of claims 26-34, further comprising an external communication module operatively coupled to one or both of the medication management device and the controller.

36. The system of claim 35, wherein the external communication module is wireless.

37. The system of claim 36, wherein the wireless external communication module comprises a WiFi radio.

38. The system of claim 36, wherein the wireless external communication module comprises a WiFi radio and a BLUETOOTH® radio.

39. A method of managing the use of a medication comprising:
providing a medication management system comprising a medication management device comprising one or more sensors and a controller comprising a user interface,
interfacing one or more containers of medication within the medication management device; and
monitoring one or more environmental parameters of the medication management device.

40. The method of claim 39, further comprising configuring the controller with a dosing schedule for the medication.

41. The method of claim 39, further comprising configuring the controller with predetermined threshold values for measurements from the one or more sensors.

42. The method of claim 40, comprising providing an alert to a user upon detection of a deviation from the medication dosing schedule.

43. The method of claim 42, comprising providing an alert to a user based on a measurement from the one or more sensors exceeding the predetermined threshold.

44. The method of claim 42 or 43, wherein the user interface of the controller displays information pertaining to the deviation from the medication dosing schedule or measurement from the one or more sensors exceeding the predetermined threshold to the user.

45. The method of any of claims 42-44, further comprising providing an alert to one or more third parties based on a measurement from the one or more sensors exceeding the predetermined threshold.

46. The method of any of claims 42-44, further comprising providing an alert to one or more third parties based on upon detection of a deviation from the medication dosing schedule.

47. The method of any of claims 42-46, wherein the medication management device is connectable to an individual container of medication.

48. The method of any of claims 42-46, wherein the medication management system is constructed and arranged to store one or more containers of medication.
